(19) United States
(12) Patent Application Publication
(10) Pub. No.: US 2006/0235234 A1
(43) Pub. Date: Oct. 19, 2006

(54) New route to alpha-tocopherol, alpha-tocopheryl alkanoates and precursors thereof

(76) Inventors: Werner Bonrath, Freiburg (DE); Manfred Breuninger, Bad Sackingen (DE); Gregory Malaise, Saint-Louis (FR); Thomas Netscher, Bad Krozingen (DE)

Correspondence Address: NIXON & VANDERHYE, PC, 901 NORTH GLEBE ROAD, 11TH FLOOR, ARLINGTON, VA, 22203, US

(21) Appl. No.: 10/571,252

(22) PCT Filed: Sep. 2, 2004
(86) PCT No.: PCT/EP04/09748

§ 371 Date: Apr. 13, 2006

(30) Foreign Application Priority Data
Sep. 15, 2003 (EP) 03020873.0

(51) Int. Cl. C07F 15/00; C07F 7/00; C07D 311/04

(52) U.S. Cl. 549/411; 556/136; 556/466; 548/101

(57) ABSTRACT

The present invention is concerned with a novel process for the manufacture of (E/Z)-4-alkanoyloxy-3,5,6-trimethyl-2-phytylphenyl esters and silyl ethers, precursors of α-tocopherol and α-tocopheryl alkanoates, by cross-metathesis reaction of 2-alkenyl-3,5,6-trimethylhydroquinone dialkanoates or 4-alkanoyloxy-2-alkenyl-3,5,6-trimethylphenyl silylethers with 2,6,10,14-tetramethylpentadecene or a phytol derivative, e.g. phytyl acetate, in the presence of a cross-metathesis catalyst. As the cross-metathesis catalyst especially ruthenium metal carbene complexes are suitable which possess (a) ruthenium metal center(s), have an electron count of 16 or 18 and are penta- or hexa-coordinated. A further object of the invention is a process for the manufacture of α-tocopherol and α-tocopheryl alkanoates comprising this reaction.

The present invention is concerned with a novel process for the manufacture of (E/Z)-4-alkanoyloxy-3,5,6-trimethyl-2-phytylphenyl esters and silyl ethers, precursors of α-tocopheryl alkanoates and α-tocopherol, by cross-metathesis reaction of 2-alkenyl-3,5,6-trimethylhydroquinone dialkanoates or 4-alkanoyloxy-2-alkenyl-3,5,6-trimethylphenyl silylethers with 2,6,10,14-tetramethylpentadecene or a phytol derivative, e.g. phytyl acetate, in the presence of a cross-metathesis catalyst. A further object of the invention is a process for the manufacture of α-tocopheryl alkanoates and α-tocopherol comprising this reaction step.

As is known, (all-rac)-α-tocopherol (or as it has mostly been denoted in the prior art, “d,l-α-tocopherol”) is a diastereoisomeric mixture of 2,5,7,8-tetramethyl-2-(4′,8′,12′-trimethyl-tridecyl)-6-chromanol (α-tocopherol), which is the most biologically active and industrially most important member of the vitamin E group. Often the acetate or another alkanoate of α-tocopherol is produced since it is more stable and more convenient to handle in contrast to α-tocopherol which is labile against oxidative conditions.

Many processes for the manufacture of “d,l-α-tocopherol” (referred to as such in the literature reviewed hereinafter) and its acetate are described in literature, of which some examples are discussed below. They all have in common that α-tocopherol or its acetate are produced by the reaction of trimethylhydroquinone (TMHQ)/trimethylhydroquinone acetate (TMHQA) with isophytol (IP), phytol (PH) or its derivatives in the presence of a catalyst or catalyst system and in a solvent or solvent system.

According to EP 0 100 471 e.g. the reaction of TMHQ with IP or PH is carried out in the presence of a Lewis acid, e.g. ZnCl₂, BF₃ or AlCl₃, a strong acid, e.g. HCl, and an amine or an amine salt of a non-oxidizing protic acid as the catalyst system.

EP-A 0 658 552 discloses a process for the preparation of α-tocopherol and derivatives thereof, wherein fluorosulfonates [M(RSO₃)₃], nitrates [M(NO₃)₃] and sulfates [M₂(SO₄)₃] are used as the catalysts with M representing a Sc, Y or lanthanide atom, and R representing fluorine, a fluorinated lower alkyl or an optionally single or multiple fluorinated aryl. The reaction is carried out in a solvent which is inert to the catalyst and the starting materials, TMHQ and allyl alcohol derivatives or alkenyl alcohols, examples of the solvent being aromatic hydrocarbons, linear and cyclic ethers, esters and chlorinated hydrocarbons.

According to EP-B 0 694 541 a carbonate ester, a lower fatty acid ester or a mixed solvent of a non-polar solvent and a lower C₁₋₅-alcohol is used as solvent for the preparation of α-tocopherol starting with TMHQ and (iso)phytol or phytol derivatives. As the catalyst a mineral acid, a Lewis acid, an acidic ion exchange resin or a triflate, nitrate or sulfate of Sc, Y or a lanthanid element is used.

In the process of EP-A 1 180 517 TMHQ and IP or PH are reacted in the presence of a bis-(perfluorinated hydrocarbyl sulphonyl)imide or a metal salt thereof to obtain α-tocopherol. Solvents for this reaction are polar organic solvents such as aliphatic and cyclic ketones, aliphatic and cyclic esters and carbonates, and non-polar organic solvents such as aliphatic and aromatic hydrocarbons or mixtures thereof.

The reaction of TMHQ/TMHQA with isophytol, phytol or an (iso)phytol derivative has the disadvantage of the formation of by-products such as phytadienes and benzofurans. The separation of these by-products from α-tocopherol and its esters such as the acetate, respectively, is rather difficult.

The object of the present invention is to provide a process for the manufacture of (all-rac)α-tocopheryl alkanoates, which are stable against oxidative conditions, α-tocopherol, and precursors thereof, whereby the production of benzofurans/phytadienes is avoided. Furthermore the catalyst used should have no, or at least a much reduced, corrosive action.

In one aspect the present invention is related to a process for the manufacture of compounds represented by the following formula III, so called (E/Z)-4-alkanoyloxy-3,5,6-trimethyl-2-phytyl-phenyl ester (=(E/Z)-2-phytyl-3,5,6-trimethylhydroquinone dialkanoate) or (E/Z)-4-alkanoyloxy-3,5,6-trimethyl-2-phytyl-phenyl silyl ether, 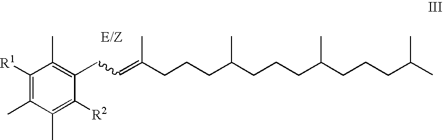
wherein R¹ and R² are as defined below,
by reacting
a) a compound represented by the following formula I, so-called 4-alkanoyloxy-2-alkenyl-3,5,6-trimethylphenyl ester (=2-alkenyl-3,5,6-trimethylhydroquinone dialkanoate; if R²═C₂₋₅-alkanoyloxy) or 4-alkanoyloxy-2-alkenyl-3,5,6-trimethylphenyl silyl ether (if R²═OSiR⁶R⁷R⁸), 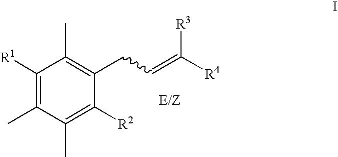
   wherein R¹ is C₂₋₅-alkanoyloxy,

R² is C₂₋₅-alkanoyloxy or OSiR⁶R⁷R⁸, wherein R⁶, R⁷ and RB are independently from each other C₁₋₆-alkyl or phenyl,

R³ and R⁴ are independently from each other H or C₁₋₅-alkyl, with the proviso that at least one of R³ and R⁴ is not H, with
b) a compound represented by the following formula II, 2,6,10,14-tetramethylpentadecene (if R⁵H) or a phytol derivative (if R⁵═CH₂R⁹),

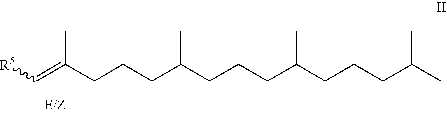

wherein R⁵ is H or CH₂R⁹, wherein R⁹ is formyloxy, C₂₋₅-alkanoyloxy, benzoyloxy, C₁₋₅-alkoxy or OSiR⁶R⁷R⁸ as defined above,

in the presence of a cross-metathesis catalyst.

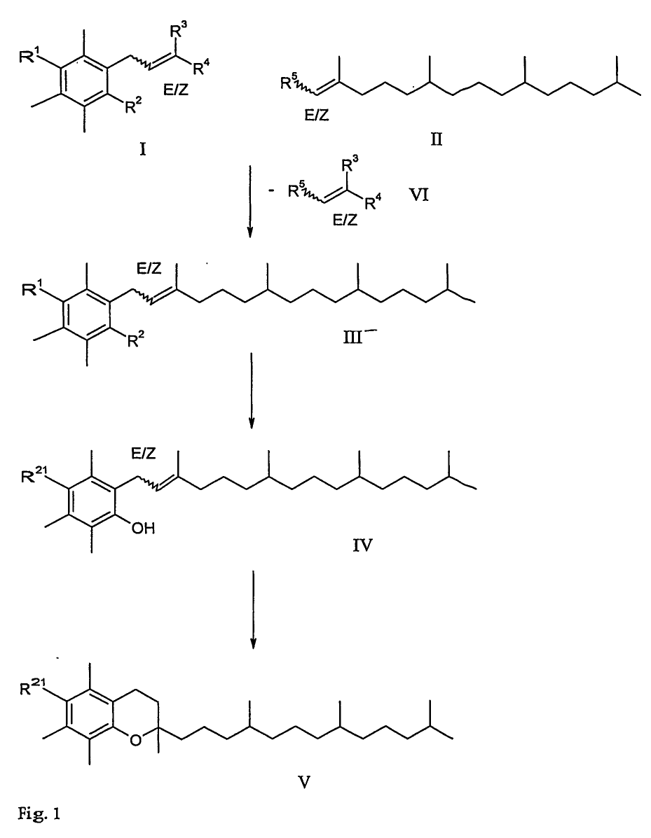

These so-called (E/Z)-4-alkanoyloxy-3,5,6-trimethyl-2-phytyl-phenyl ester (=(E/Z)-2-phytyl-3,5,6-trimethylhydroquinone dialkanoate) and (E/Z)-4-alkanoyloxy-3,5,6-trimethyl-2-phytyl-phenyl silyl ether, respectively, are 2-phytyl-3,5,6-trimethylhydroquinone derivatives and suitable precursors for α-tocopherol and x-tocopheryl alkanoates represented by the formula V as shown in FIG. 1. The reaction of a compound of formula I with a compound of formula II is a cross-metathesis reaction. The compounds represented by formula VI (see FIG. 1) are produced as byproducts. They can easily be removed by column chromatography or distillation.

Concerning the substituent R¹: The term “C₂₋₅-alkanoyloxy” covers linear C₂₋₅-alkanoyloxy and branched C₄₋₅-alkanoyloxy. R¹ is preferably acetyloxy or pivaloyloxy, more preferably it is acetyloxy.

Concerning the substituent R²: The expression “C₂₋₅-alkanoyloxy” incorporates linear C₂₋₅-alkanoyloxy and branched C₄₋₅-alkanoyloxy. The term C₁₋₆-alkyl encloses linear C₁₋₆-alkyl and branched C₃₋₆-alkyl.

Preferably R² is OSiR⁶R⁷R⁸, more preferably R² is OSiR⁶R⁷R⁸ with R⁶, R⁷ and R⁸ being C₁₋₆-allyl, whereby the number of the C atoms of all three substituents R⁶, R⁷ and R⁸ together is at least 6. Most preferred R² is OSi^{t}BuMe₂ (Me=methyl, ^{t}Bu=tert-butyl), OSiⁱPr₃ (ⁱPr=iso-propyl) or OSiBu₃ (Bu=n-butyl).

Concerning the substituents R³ and R⁴: The expression “C₁₋₅-alkyl” embraces linear C₁₋₅-alkyl and branched C₃₋₅-alkyl. Preferably R³ and R⁴ are independently from each other C₁₋₅-alkyl, more preferably they are both identical C₁₋₅-alkyl, most preferably they are both methyl.

Concerning the substituent R⁵: The expression “C₂₋₅-alkanoyloxy” incorporates linear C₂₋₅-alkanoyloxy and branched C₄₋₅-alkanoyloxy and the expression “C₁₋₅-alkoxy” covers linear C₁₋₅-alkoxy and branched C₃₋₅-alkoxy.

R⁵ is preferably H or CH₂R⁹, wherein R⁹ is formyloxy, C₂₋₅-alkanoyloxy, benzoyloxy and OSiR⁶R⁷R⁸ as defined above. More preferably R⁵ is H or CH₂R⁹ with R⁹ being formyloxy, C₂₋₅-alkanoyloxy or benzoyloxy, most preferably R⁵ is H.

The Cross-Metathesis Catalyst

Preferably the cross-metathesis catalyst used in the process according to the invention is a ruthenium compound used in homogeneous catalysis. Homogeneous catalysis means that the reaction mixture is monophasic during the catalyzed reaction.

More preferably the ruthenium compound is a ruthenium metal carbene complex possessing (a) ruthenium metal center(s), having an electron count of 16 and being penta-coordinated or a ruthenium metal carbene complex possessing (a) ruthenium metal center(s), having an electron count of 18 and being hexa-coordinated. Preferred is a ruthenium metal carbene complex possessing a ruthenium metal center, having an electron count of 16 and being penta-coordinated. It has to be kept in mind that these are the forms in which the catalysts are present before the reaction, so-called “precatalysts”. The real “ccatalytic” species is formed in situ during the reaction, of which the structure is not known.

“Penta-coordinated” in this context does not necessarily mean that there are five ligands per Ru metal center in the complex. It is also possible that one ligand provides two coordination sites, i.e. that the complex contains four ligands per Ru metal center. The same applies for the term “hexa-coordinated”. Hexa-coordinated Ru-complexes might contain five or six ligands, one of the five ligands providing two coordination sites, a so-called bidentate ligand.

More preferred examples for such ruthenium compounds are the ruthenium metal carbene complexes represented by the following formulae VIIa, VIIb and VIIc: 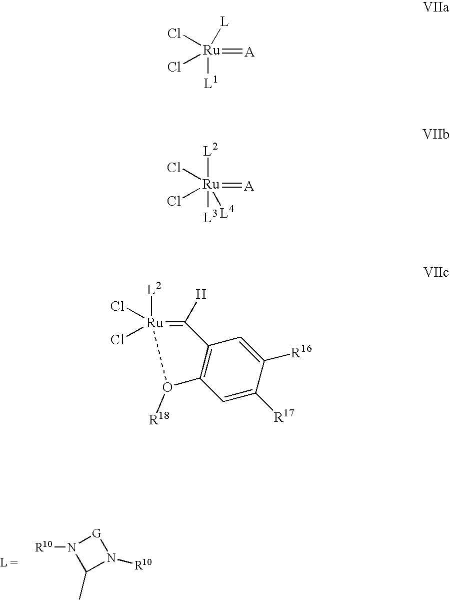
wherein R¹⁰ is an optionally single or multiple C₁₋₅-alkylated and/or C₁₋₅-alkoxylated phenyl,
G is ethane-1,2-diyl, ethylene-1,2-diyl, cyclohexane-1,2-diyl or 1,2-diphenylethane-1,2-diyl,
L¹ is PR¹¹R¹²R¹³, wherein R¹¹, R¹² and R¹³ are independently from each other C₁₋₈-alkyl, phenyl or tolyl,
A is CH₂, C(H)aryl, C(H)R¹⁴, C═C(R⁴)₂, C═C(H)Si(R¹⁵)₃, C(H)—C(H)═C(R¹⁴)₂, C═C(H)(phenyl), C(H)—C(H)═C(phenyl)₂ or C═C═C(phenyl)₂,
wherein “aryl” is an optionally single or multiple C₁₋₅-alkylated and/or halo genated phenyl, R¹⁴ is C₁₋₄-alkyl, R¹⁵ is C₁₋₆-alkyl or phenyl,
L² is L or L¹,
L³ and L⁴ are independently from each other pyridyl or 3-halopyridyl, wherein halo signifies Br or C¹,
R¹⁶ and R¹⁷ are both H or form together a fused benzene ring, and R¹⁸ is C₁₋₅-alkoxy.

Concerning the substituent R¹⁰: Preferred examples for an optionally single or multiple C₁₋₅-alkylated and/or C₁₋₅-alkoxylated phenyl are phenyl, 2,6-dimethylphenyl, 2,3,6-trimethylphenyl, 2,4,6-trimethylphenyl, 2,6-dimethyl-4-methoxy-phenyl, 2-isopropylphenyl, 2,6-diisopropylphenyl and 2-isopropyl-6-methylphenyl. More preferred examples for R¹⁰ are 2,6-dimethylphenyl, 2,4,6-trimethylphenyl and 2,6-diisopropylphenyl.

Concerning the substituent G: Preferably G is ethane-1,2-diyl.

Concerning the substituent L¹: The term “C₁₋₈-alkyl” includes linear C₁₋₈-alkyl, branched C₃₋₈-alkyl and C₅₋₈-cycloalkyl. Preferably L¹ is P(R¹¹)₃, wherein R¹¹ is linear C₁₋₈-alkyl, C₅₋₈-cycloalkyl or phenyl. More preferably L¹ is P(C₆H₁₁)₃ (“C₆H₁₁”=cyclohexyl), P(C₅H₉)₃ (“C₅H₉”=cyclopentyl) or PPh₃ (“Ph”=phenyl).

Concerning the substituent A: The term “halogenated” means fluorinated, chlorinated or brominated, whereby chlorinated is preferred. Preferred examples for an optionally single or multiple C₁₋₅-alkylated and/or halogenated phenyl are phenyl, 4-chlorophenyl, 2,6-dimethylphenyl, 2,3,6-trimethylphenyl, 2,4,6-trimethylphenyl, 2,6-dimethyl-4-methoxyphenyl, 2-isopropylphenyl, 2,6-diisopropylphenyl and 2-isopropyl-6-methylphenyl. The expression “C₁₋₄-alkyl” (substituent R¹⁴) includes linear C₁₋₄-alkyl as well as branched C₃₋₄-alkyl. The expression “C₁₋₆-alkyl” (substituent R¹⁵) includes linear C₁₋₆-alkyl as well as branched C₃₋₆-alkyl.

Preferably A is C(H)CH₃, C(H)CH₂CH₃, C(H)(phenyl), C(H)(4-chlorophenyl), C═C(H)(phenyl), C═C(H)Si(CH₃)₃, C(H)—C(H)═C(Me)₂ and C(H)—C(H)═C(phenyl)₂. More preferably A is C(H) (phenyl), C(H)—C(H)═C(Me)₂ and C(H)—C(H)═C(phenyl)₂. Most preferably A is C(H)(phenyl).

Concerning the substituents L³ and L⁴: Preferably L³ and L⁴ are both identical. More preferably they are both 3-bromopyridyl.

Concerning the substituents R¹⁶ and R¹⁷: Preferably they are both H.

Concerning the substituent R¹⁸: The term “C₁₋₅-alkoxy” includes linear C₁₋₅-alkoxy as well as branched C₃₋₅-alkoxy. Preferably R¹⁸ is isopropoxy or methoxy, more preferably R¹⁸ is isopropoxy.

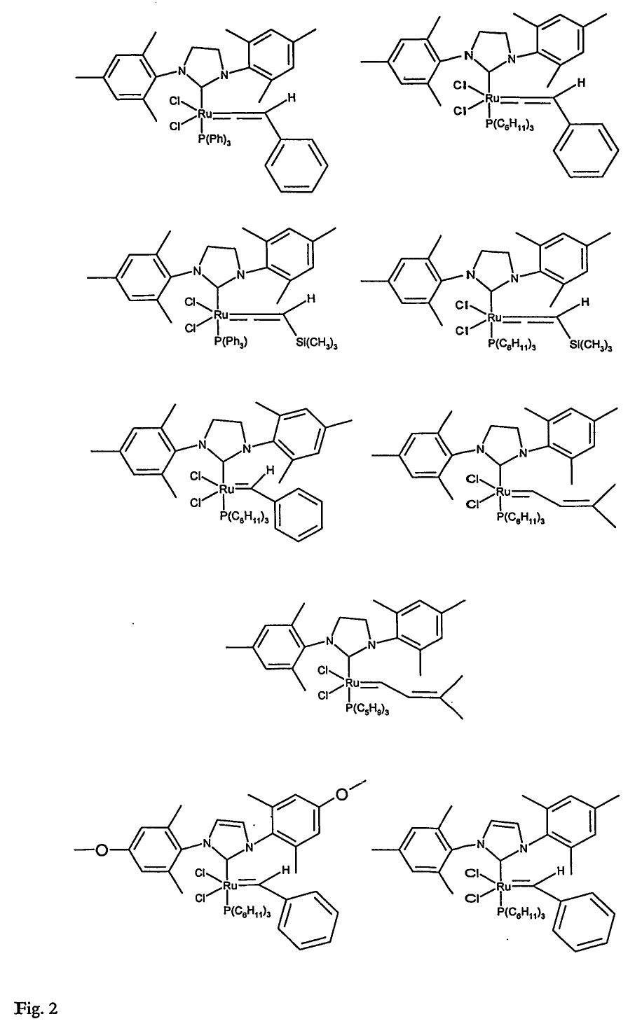

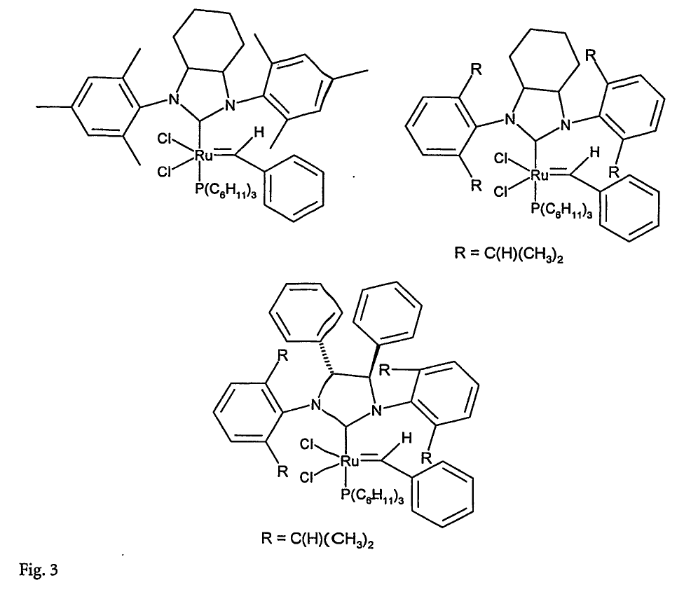

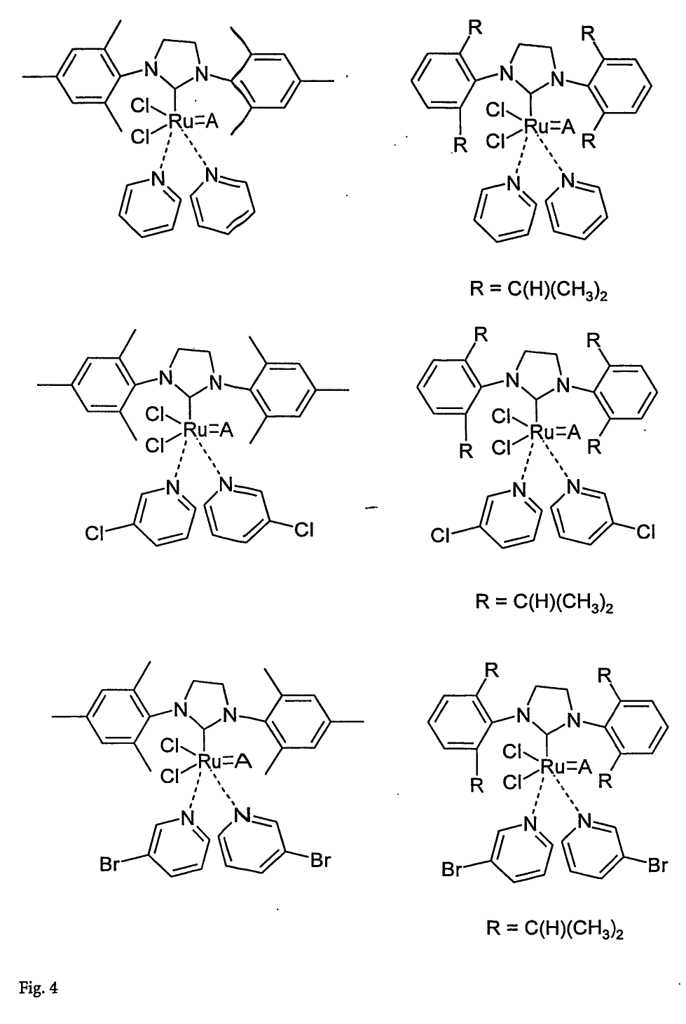

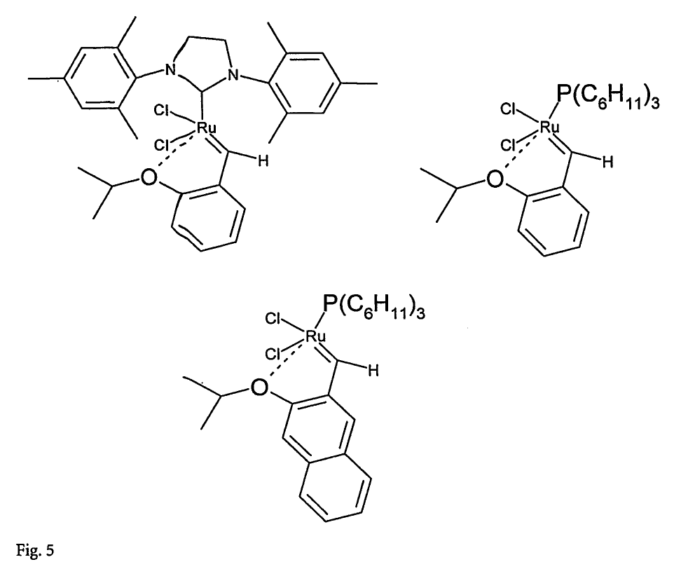

Preferred examples for complexes represented by the formula VIIa are illustrated in FIGS. 2 and 3. Preferred examples for complexes represented by the formula VIIb are illustrated in FIG. 4 (A is C(H)CH₃, C(H)CH₂CH₃, C(H)(phenyl), C(H)(4-chlorophenyl), C═C(H)(phenyl), C═C(H)Si(CH₃)₃, C(H)—C(H)═C(Me)₂ and C(H)—C(H)═C(phenyl)₂). Preferred examples for complexes represented by the formula VIIc are illustrated in FIG. 5.

The most preferred cross-metathesis catalyst used in the process according to the invention is the following ruthenium metal carbene complex of formula VIII: 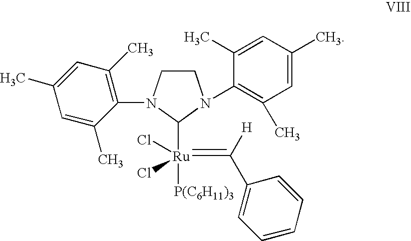
Synthesis of the Catalyst

The synthesis of the ruthenium carbene complexes represented by the formulae VIIa, VIIb, VIIc and VIII is e.g. described by P. Schwab, M. B. France, J. W. Ziller and R. H. Grubbs in Angew. Chem. Int. Ed. Engl. 1995, 34(18), 2039-2041; by M. Scholl, S. Ding, C. W. Lee and R. H. Grubbs in Organic Letters 1999, 1(6), 953-956 (see especially footnote 16); by S. B. Garber, J. S. Kingsbury, B. L. Gray and A. H. Hoveyda in J. Am. Chem. Soc. 2000, 122, 8168-8179; by J. Huang, E. D. Stevens, S. P. Nolan and J. L. Petersen in J. Am. Chem. Soc. 1999, 121, 2674-2678 (see especially page 2678); by M. Scholl, T. M. Trnka, J. P. Morgan and R. H. Grubbs in Tetrahedron. Lett. 1999, 40, 2247-2250 (see especially note 13); by S. T. Nguyen, L. K. Johnson and R. H. Grubbs in J. Am. Chem. Soc 1992, 114, 3974-3975 and the supplementary material thereto; by T. Opstal and F. Verpoort in Synlett 2003, 3, 314-320 (see especially reference 16); by M. S. Sanford, M. Ulman and R. H. Grubbs in J. Am. Chem. Soc. 2001, 123, 749-750 (see especially the supplementary material thereto); by A. K. Chatterjee and R. H. Grubbs in Organic Letters 1999, 1(11), 1751-1753; by A. K. Chatterjee, J. P. Morgan, M. Scholl and R. H. Grubbs in J. Am. Chem. Soc. 2000, 122, 3783-3784; and by J. P. Morgan and R. H. Grubbs in Organic Letters 2000, 2(20), 3153-3155 (footnote 13).

Synthesis of the Starting Material

2-Alkenyl-3,5,6-trimethylhydroquinone 1-acetate can be prepare d by O-alkylation of 2,3,6-trimethylhydroquinone 1-acetate followed by a rearrangement analogous to the processes as e.g. described by Y. Tanada, K. Mori in Eur. J. Org. Chem. 2003, 848-854 (see especially scheme 5 and the preparation of compound 19 on page 852 and 853); by J. C. Gilbert, M. Pinto in J. Org. Chem. 1992, 57, 5271-5276; in EP-A 0 345 593 (see especially reference examples 1 and 2 on page 6/7); or by N. Al-Maharik, N. G. Botting in Tetrahedron 2003, 59, 4177-4181 (see especially chapter 3.1.2 and 3.1.3). The other 2-alkenyl-3,5,6-trimethylhydroquinone 1-alkanoates can be prepared analogously or by a Friedel-Crafts alkylation (see example A).

The starting material for those, the 2,3,6-trimethylhydroquinone 1-alkanoates (=4-alkanoyloxy-2,3,5-trimethylphenols) such as 2,3,6-trimethylhydroquinone 1-acetate, may be obtained e.g. by selective hydrolysis of the dialkanoates such as 2,3,5-trimethylhydroquinone diacetate as described in EP-A 1 239 045.

2,3,5-Trimethylhydroquinone diacetate can be prepared e.g. by the acid catalyzed rearrangement of ketoisophorone in the presence of acetic anhydride or another acetylation agent as described in EP-A 0 850 910, EP-A 0 916 642, EP-A 0 952 137 or EP-A 1 028 103. The other alkanoates can be prepared by acylation of TMHQ.

4-alkanoyloxy-2-alkenyl-3,5,6-trimethylphenyl silyl ethers, compounds represented by the formula I with R²═OSiR⁶R⁷R⁸, can be prepared by silylation of 2-alkenyl-3,5,6-trimethylhydroquinone 1-alkanoate with ClSiR⁶R⁷R⁸ according to standard procedures for the silylation of alkohols and as e.g. described by E. J. Corey and A. Venkateswarlu in J. Am. Chem. Soc. 1972, 94(17), 6190-6191.

2-Alkenyl-3,5,6-trimethylhydroquinone dialkanoates were synthesised by acylation of 2-Alkenyl-3,5,6-trimethylhydroquinone 1-alkanoate in the presence of an acylating agent.

2,6,10,14-Tetramethylpentadecene may be obtained according to the procedure disclosed of K. Sato, S. Mizuno, M. Hirayama in J. Org. Chem. 1967, 32, 177-180 (see especially page 180).

The phytol derivatives, compounds b) represented by the formula II with R⁵═CH₂R⁹, can be produced by conventional processes for preparing phytyl esters, phytyl silyl ethers and phytyl ethers known to the person skilled in the art. Processes for their manufacture are e.g. described in EP-A 0 004 889 or in FR-A 2 627 384.

The 3-alkenyl-2,5,6-trimethylhydroquinone derivatives of formula I as well as the phytol derivatives of formula II with R⁵═CH₂R⁹ can be used as E/Z-mixture as well as in pure E- or pure Z-form. In both cases preferred is the use of the E/Z-mixtures, since the E/Z ratio of these starting materials is not maintained in the resulting product of formula III and the later following ring closure is independent from this ratio (see FIG. 1)

Cross-Metathesis Reaction

The catalysts, especially those represented by the formulae VIIa, VIIb, VIIc and VIII, which can be obtained e.g. according to the processes described in the literature cited above or are also commercially available. Conveniently they are used as solution, whereby as solvent that solvent is used in which the reaction is carried out. The concentration of the solution is not critical. Conveniently the concentration of the solution is from about 0.05 to about 2% by weight, preferably from about 0.1 to about 1% by weight, more preferably from about 0.4 to about 0.6% by weight, based on the total weight of the solution. If the reaction is carried out essentially in the absence of an additional solvent, the catalyst is used as such.

Conveniently the reaction is carried out in the absence or presence of an aprotic organic solvent and essentially in the absence of water and protic (in) organic solvents. “Essentially” in this context means that the amount of water, protic (in) organic solvents and additional solvent, respectively, is lower than 0.05 mol %, preferably lower than 0.01 mol %, more preferably lower than 0.005 mol %-referred to the total amount of solvent.

If the reaction is carried out in an additional aprotic organic solvent, especially dialkyl ethers R¹⁹—O—R²⁰, wherein R¹⁹ and R²⁰ are independently from each other linear C₁₋₄-alkyl or branched C₃₋₈-alkyl, R¹⁹—O—R²⁰ preferably being methyl t-butyl ether, diethyl ether or 2,2-dimethylpropyl methyl ether; tetrahydrofuran; tetrahydropyran; 1,4-dioxane; methylene chloride; chloroform; cumene (=iso-propylbenzene) and an optionally once, twice or thrice methylated arylene such as benzene, toluene, 1,2-xylene, 1,3-xylene, 1,4-xylene, mesitylene, pseudocumene, hemellitene or mixtures thereof are used.

More preferably the aprotic organic solvent is tetrahydrofuran, methylene chloride, chloroform, toluene or a mixture thereof. The most preferred aprotic organic solvent is toluene.

The molar ratio of the compound a) of the formula I to the compound b) of the formula II in the reaction mixture conveniently varies from about 1:10 to about 10:1, preferably from about 1:5 to about 5:1, more preferably from about 1:3 to about 1:2.5. Most preferably compound b) is used in excess. If an excess of compound b) of formula II, 2, 6,0,14-tetramethylpentadecene or a phytol derivative, is used, non-reacted material can be recycled after termination of the reaction and separation of the product by column chromatography. The same applies if an excess of compound a) is used. In general also a mixture of the non-reacted starting materials, compounds a) and b), can be recycled.

The amount of the cross-metathesis catalyst used, especially of the formulae VIIa, VIIb, VIIc and VIII, is based on the amount of compound a) or b), whichever is used in the lesser molar amount. Usually the relative amount of the catalyst to the amount of compound a) or b), whichever is used in the lesser molar amount, preferably to the amount of compound a) used in the lesser amount, is from about 0.0001 to about 20 mol %, preferably from about 1.0 to about 10 mol %, more preferably from about 2 to about 5 mol. %. In this context the expression “amount of catalyst” is to be understood as referring to the amount of the pure catalyst present, even though the catalyst may be impure and/or in the form of an adduct with a solvent.

The amount of the aprotic organic solvent used is conveniently from about 3 to about 15 ml, preferably from about 4 ml to about 10 ml, more preferably from about 4.5 ml to 8 ml, based on 1 mmol of compound a) or b), whichever is used in the lesser amount.

The reaction temperature is dependent from the solvent/solvent mixture used. Conveniently it ranges from about 10° C. to about 120° C., preferably from about 30° C. to about 100° C., more preferably from about 40° C. to about 85° C.

The pressure under which the reaction is carried out is not critical, but dependent from the temperature and the solvent/solvent mixture used. The reaction is conveniently carried out at atmospheric pressure, but when solvents/solvent mixtures with a boiling point below the reaction temperature are used, pressure must be applied. Essentially in the absence of an additional solvent the reaction is carried out preferably at reduced pressure, especially at a pressure below 100 mbar, a pressure below 40 mbar being even more preferred.

Moreover, the process is conveniently carried out under an inert gas atmosphere, preferably gaseous nitrogen or argon.

The process in accordance with the invention can be carried out batchwise or continuously, and in general operationally in a very simple manner, e.g. by adding a mixture of compounds a) and b)—as such or dissolved in the aprotic organic solvent such as mentioned above, preferably as solution—continuously to a mixture of the catalyst and the aprotic organic solvent.

After completion of the addition and an appropriate subsequent reaction period the isolation of the product and its purification if required, can be effected by procedures conventionally used in organic chemistry.

The present invention provides a new route to (E/Z)-2-phytyl-3,5,6-trimethylhydroquinone dialkanoate, (E/Z)-4-alkanoyloxy-3,5,6-trimethyl-2-phytyl-phenyl silyl ether (compounds of formula III), α-tocopherol and α-tocopheryl alkanoates (compounds of formula V, wherein R²¹ is OH and C₂₋₅-alkanoyloxy, respectively) (see FIG. 1). This process has the advantage of avoiding the production of benzofurans/phytadienes, formed during conventional synthesis of α-tocopherol and its derivatives and difficult to remove from the product. A further advantage of the process in accordance with the invention is, in addition to the work at lower temperatures compared with conventional α-tocopherol (alkanoate) production processes, the avoidance of corrosion.

Another aspect of the present invention is a process for the manufacture of α-tocopherol and α-tocopheryl alkanoates represented by the following formula V 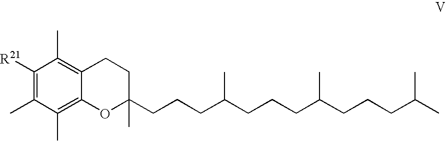
comprising the following steps:
i) reacting of a compound represented by the following formula I 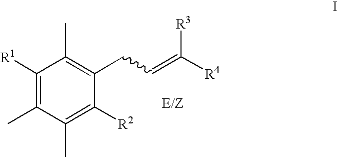
with a compound represented by the following formula II 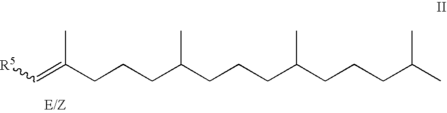
to a compound represented by the following formula III 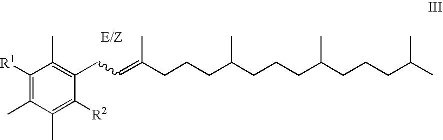
in the presence of a cross-metathesis catalyst,
ii) converting the compound represented by the formula III and obtained in step i) to (E/Z)-3-phytyl-2,5,6-trimethylhydroquinone (R²¹═OH) or a (E/Z)-3-phytyl-2,5,6-trimethylhydroquinone 1-alkanoate (R²¹═C₂₋₅-alkanoyloxy) represented by the following formula IV, and 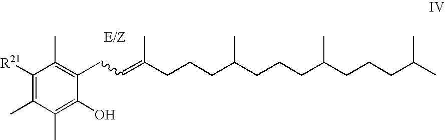
iii) subjecting the compound represented by the formula IV and obtained in step ii) to a cyclization to α-tocopherol (R²¹═OH) or an α-tocopheryl alkanoate (R²═C₂₋₅-alkanoyloxy) represented by the formula V,
   wherein R¹, R², R³, R⁴ and R⁵ are as defined above, and R²¹ is OH or R¹.

While the production of (all-rac)-α-tocopheryl alkanoates and (all-rac)-α-tocopherol is preferred, the invention is not limited to the production of those particular isomeric forms and other isomeric forms can be obtained by using 2,6,10,14-tetramethylpentadecene or a phytol derivative as the starting material in the appropriate isomeric form. Thus, (RS,R,R)α-tocopheryl alkanoate and (RS,R,R)-α-tocopherol will be obtained when using (R,R)-2,6,10,14-tetramethylpentadecene or a (R,R)-phytol derivative as starting material.

Step i) is carried out as described above. The steps ii) and iii) are further described in more detail in the following.

Step ii)

Step ii) is depending on the substituent R² of compound III. If R² is OSiR⁶R⁷R⁸ as defined above and R¹ is C₂₋₅-alkanoyloxy, the silyl ether might be selectively cleaved in presence of the ester to yield (E/Z)-3-phytyl-2,5,6-trimethylhydroquinone 1-alkanoate. The cleavage of silylethers is e.g. carried out as described by S. V. Ankala and G. Fenteany in Tetrahedron Lett. 2002, 43, 4729-4732.

If R¹ and R² are both C₂₋₅-alkanoyloxy, both ester groups are cleaved under acidic or basic conditions or by hydrogenolysis or as e.g. described by C. Ramesh, G. Mahender, N. Ravindranath and B. Das in Tetrahedron 2003, 59, 1049-1054 and the references cited therein. The thus obtained (E/Z)-3-phytyl-2,5,6-trimethylhydroquinone (formula IV with R²═OH) can be used in step iii) to obtain α-tocopherol.

Step iii)

The ring closure of (E/Z)-3-phytyl-2,5,6-trimethylhydroquinone and (E/Z)-3-phytyl-2,5,6-trimethylhydroquinone 1-alkanoate, respectively, in accordance with the invention can be effected by their treating with an acid catalyst in the presence or absence of a solvent according to/analogous to the procedure described in WO 03/37883 for the ring closure of (E/Z)-3-phytyl-2,5,6-trimethylhydroquinone. The content of WO 03/37883 is incorporated herein.

Advantageously the thus obtained α-tocopherol (formula V with R²¹═OH) is transferred into its alkanoate (formula V with R²¹═C₂₋₅-alkanoyloxy) by acylation as e.g. described in U.S. Pat. No. 2,723,278 and U.S. Pat. No. 6,444,098, since the alkanoates are more stable than α-tocopherol itself. Therefore the process where compounds of formula I with R²═OSiR⁶R⁷R⁸, wherein R⁶, R⁷ and R⁸ are as defined above, are used is preferred, since it produces the more stable α-tocopheryl alkanoates via (E/Z)-3-phytyl-2,5,6-trimethylhydroquinone 1-alkanoates.

The following examples illustrate the invention in more detail, but are not intended to limit its scope in any way.

### EXAMPLES

The structure of the products was confirmed with ¹H nuclear magnetic resonance spectroscopy (¹H NMR), mass spectroscopy (MS), infrared spectroscopy (IR) and elemental analysis. Their purity was checked with gas chromatography (GC).

### Examples A-I

### Synthesis of the Starting Material

### Example A

### Synthesis of 3-(3′-methyl-2′-butenyl)-2,5,6-trimethylhydroquinone 1-acetate

A 1.5 l european-style three neck flask, equipped with a mechanical stirrer, a connection to inert gas and a 50 ml dropping funnel was charged with 515 mmol (100 g) of 2,3,6-trimethylhydroquinone 1-acetate, 670 mmol (57.7 g; 70 ml) of 3-methyl-butene-3-ol and 1 l of methylene chloride and cooled in an icebath to 0° C. and flushed with argon. The dropping funnel was charged with ca. 260 mmol (32.5 ml) of BF₃.Et₂O (˜48%; Fluka, product number: 15720). This solution was added dropwise under stirring and ice cooling during 2 hours. After another 30 min the reaction mixture was poured on 1 liter of an aqueous 5% by weight sodium bicarbonate solution and stirred for 1 hour at 22° C. The organic phase was separated, washed neutral with an aqueous 5% by weight sodium bicarbonate solution and brine. The aqueous phases were extracted twice with 200 ml of methylene chloride. The combined organic phases were dried over sodium sulfate, filtered and concentrated in vacuo to give 146.4 g of a crude product. Recristallisation from 250 ml of boiling n-hexane gave 95.3 g of a white cristalline product of 97.7% purity (GC area) melting at 110° C. The yield is 69%-based on 2,3,6-trimethylhydroquinone 1-acetate.

### Example B

### Synthesis of 3-(3′-methyl-2′-butenyl)-2,5,6-trimethylhydroquinone diacetate

(Formula I with R¹═R²═OC(O)CH₃, R³═R⁴═CH₃)

3-(3′-Methyl-2′-butenyl)-2,5,6-trimethylhydroquinone 1-acetate is acetylated with acetic anhydride in the presence of catalytic amounts of N,N-dimethylaminopyridine according to standard procedures known to the person skilled in the art to give 3-(3′-methyl-2′-butenyl)-2,5,6-trimethylhydroquinone diacetate.

### Example C

### Synthesis of 4-acetyloxy-2-(3′-methyl-2′-butenyl)-3,5,6-trimethylphenyl tributylsilyl ether

(formula I with R¹═OC(O)CH₃, R²═OSiBu₃, R³═R⁴═CH₃)

A schlenk tube equipped with a magnetic stirrer and placed under argon was charged with 2.0 mmol (515 mg) of 3-(3′-methyl-2′-butenyl)-2,5,6-trimethylhydroquinone 1-acetate and 6 mL of dry tetrahydrofurane. To this solution were added dropwise via syringes successively 2.0 mmol (280 μL) of triethylamine and 2.0 mmol (335 μL) of n-tributylchlorosilane. The resulting solution was heated to 50° C. while a white precipitate was rapidly formed. After 18 hours at 50° C. the solvent was evaporated in vacuo and the crude product was purified by column chromatography over silica gel using a mixture of diethylether and hexane (v/v=1:9) as eluent. 11.0 mmol (510 mg) of 4-acetyloxy-2-(3′-methyl-2′-butenyl)-3,5,6-trimethylphenyl tributylsilyl ether were isolated as a colorless oil (yield: 55% based on 3-(3′-methyl-2′-butenyl)-2,5,6-trimethylhydroquinone 1-acetate, purity: 99.3%-GC area).

### Example D

### Synthesis of 4-acetyloxy-2-(3′-methyl-2′-butenyl)-3,5,6-trimethylphenyl tert-butyldimethylsilyl ether

(formula I with R¹═OC(O)CH₃, R²═OSiMe₂^{t}Bu, R³═R⁴═CH₃)

A schlenk tube equipped with a magnetic stirrer and a placed under argon was charged with 5.0 mmol (1.31 g) of 3-(3′-methyl-2′-butenyl)-2,5,6-trimethylhydroquinone 1-acetate, 7.1 mmol (1.13 g) of tert-butyldimethylchlorosilane, 15.0 mmol (1.02 g) of imidazole and 5 mL of dry dimethylformamide (DMF). The yellow solution was stirred at 22-23° C. during 16 hours. Then, 40 mL of diethylether and 15 ml of an aqueous solution of HCl (10% by weight) were added and the organic phase was extracted thrice with 15 mL of diethylether. The combined organic phases were washed with 30 mL of a saturated aqueous solution of NaHCO₃ and dried over Na₂SO₄. After filtration, the solvent was removed in vacuo to afford an oil which was purified by column chromatography over silica gel using a mixture of diethylether and hexane (v/v=1:4) as eluent. 4.9 mmol (1.84 g) of 4-acetyloxy-2-(3′-methyl-2′-butenyl)-3,5,6-trimethylphenyl tert-butyldimethylsilyl ether were isolated as a slightly yellow oil which solidified upon standing at 22-23° C. (yield: 98% based on 3-(3′-methyl-2′-butenyl)-2,5,6-trimethylhydroquinone 1-acetate, purity: 99.6%-GC area).

### Example E

### Synthesis of 2,6,10,14-tetramethylpentadecene

(Formula II with R⁵═H)

A flask, equipped with a mechanical stirrer and placed under argon, was charged with 1.80 mol (642.6 g) of triphenylmethylphosphonium bromide and 2 L of tetrahydrofurane (THF). To this suspension 1.88 mol (1.175 L) of a solution of butyllithium (1.6 mol/L in hexane) were added dropwise during 3 hours at 0° C. The resulting mixture was stirred at 0° C. for an additional hour. Then 1.50 mol (402.7 g) of 6,10,14-trimethyl-2-pentadecanone were added dropwise at 0° C. during 1 hour and the mixture was allowed to warm up to 22 to 23° C. After 2 hours, 150 mL of water were added dropwise and the resulting white suspension was filtered off over decalite. The filtrate was washed thrice with 300 mL of water and dried over Na₂SO₄. After filtration and evaporation of the solvent in vacao, the mixture was filtered off over decalite to remove white cristals and the filtrate was evaporated in vacuo. The resulting crude oil was purified by distillation under vacuum (145° C., 0.2 mbar) to give 1.20 mol (319.2 g) of 2,6,10,14-tetramethylpentadecene as a colorless oil (yield: 80% based on 6,10,14-trimethyl-2-pentadecanone; purity: 96.2%-GC area).

### Example F

### Synthesis of (E/Z)-(all-rac)-phytyl acetate

(Formula II with R⁵═CH₂OC(O)CH₃)

A mixture of 20 mmol (6.23 g) of (E/Z)-(all-rac)-phytol (E/Z ratio=72/28), 25 mmol (1.98 g) of pyridine, 20 mmol (2.04 g) of acetic anhydride and 5 mL of n-hexane was stirred at 21 to 22° C. for 16 to 18 hours. 30 mL of water were added and the resulting mixture was extracted thrice with 50 mL of diethyl ether. The organic phases were combined and washed thrice with 30 mL of aqueous HCl (10% by weight), neutralised with 50 mL of a saturated solution of NaHCO₃, washed with 50 mL of a saturated solution of NaCl and with 50 mL of water and dried over Na₂SO₄. After filtration, the solvent was removed in vacuo to afford a colorless oil which was purified by column chromatography over silica gel using a mixture of diethylether and hexane (v/v=1:4) as eluent. 5.62 g (16.6 mmol) of (all-rac)-Phytyl acetate were obtained as a colorless oil with an E/Z ratio of 2.5 (yield: 83% based on (all-rac)-phytol, purity: 98.2%-GC area).

### Example G

### Synthesis of (E)-(R,R)-phytyl acetate

Example F was repeated, but instead of (all-rac)-phytol (E)-(RR)-phytol (E/Z=99.7/0.3) was used. (E)-(R,R)-phytyl acetate (purity: 96.5%-GC area); E/Z=99.6/0.4) was obtained in a yield of 60.5%.

### Example H

### Synthesis of (E,Z)-(all-rac)-phytyl formiate (according to EP-A 0 004 889)

(formula II with R⁵═CH₂OC(O)H)

A mixture of 10 mmol (3.11 g) of (E,Z)-(all-rac)-phytol (E/Z=72/28) and 100 mmol (4.60 g) of formic acid was vigorously stirred at 60° C. for 2.5 hours. Then 30 mL of water were added to the mixture and the organic phase was extracted twice with 30 mL of diethyl ether. The combined organic phases were dried over Na₂SO₄ and after filtration, the solvent was removed in vacuo to afford a yellow oil. This oil was was purified by column chromatography over silica gel using a mixture of diethylether and hexane (v/v=5:95) as eluent. 9.0 mmol (2.92 g) of (E,Z)-(all-rac)-phytyl formiate were obtained as a colorless oil (E/Z=65/35; yield: 90% based on (all-rac)-phytol).

### Example I

### Synthesis of (E,Z)-(all-rac)-phytyl benzoate

(formula II with R⁵═CH₂OC(O)(phenyl))

A mixture of 48.6 mmol (15.02 g) of (E,Z)-(all-rac)-phytol (E/Z=72/28), 51.1 mmol (11.56 g) of benzoic anhydride and 2.4 mmol (0.30 g) of N,N-dimethylaminopyridine in 30 mL of hexane was stirred at 23 to 24° C. for 20 hours. Then 50 ml of water were added and the organic phase was extracted thrice with 50 mL of diethyl ether. The combined organic phases were washed thrice with an aqueous solution of HCl (10% by weight), neutralised with 50 mL of a saturated solution of NaHCO₃, washed with 50 mL of a saturated solution of NaCl and with 50 mL of water and dried over Na₂SO₄. After filtration, the solvent was evaporated in vacuo to afford a colorless oil and a white precipitate. This crude material was purified by column chromatography over silica gel using a mixture of ethyl acetate and hexane (v/v=5:95) as eluent. 37.2 mmol (14.80 g) of (E,Z)-(all-rac)-phytyl benzoate were isolated as a colorless oil (E/Z=68/32; yield: 76% based on (all-rac)-phytol; purity: 99.5%-GC area).

### Examples J-U

### Synthesis of (E/L)-3-phytyl-2,5,6-trimethylydroquinone derivatives

In the following examples complex VIII was used as catalyst for the cross-metathesis reactions. The results of the cross-metathesis reactions are summarized in table 1.

TABLE 1
Results of the cross-metathesis reactions (examples J-S)

| | | | | E/Z ratio |
|---|---|---|---|---|
| | | | | of the |
| Example | Starting material | product | yield | product |
| J | 3-(3′-methyl-2′-butenyl)- | (E/Z)-3-phytyl-2,5,6- | 69% | 2.5 |
| | 2,5,6-trimethylhydroquinone | trimethylhydroquinone | | |
| | diacetate + 2,6,10,14- | diacetate | | |
| | tetramethylpentadecene | | | |
| K | 3-(3′-methyl-2′-butenyl)- | (E/Z)-3-phytyl-2,5,6- | 60% | 2.3 |
| | 2,5,6-trimethylhydroquinone | trimethylhydroquinone | | |
| | diacetate + 2,6,10,14- | diacetate | | |
| | tetramethylpentadecene in | | | |
| | vacuo | | | |
| L | 3-(3′-methyl-2′-butenyl)- | (E/Z)-3-phytyl-2,5,6- | 46% | 2.1 |
| | 2,5,6-trimethylhydroquinone | trimethylhydroquinone | | |
| | diacetate + (E/Z)-(all-rac)- | diacetate | | |
| | phytyl acetate | | | |
| M | 3-(3′-methyl-2′-butenyl)- | (E/Z)-3-phytyl-2,5,6- | 50% | 2.0 |
| | 2,5,6-trimethylhydroquinone | trimethylhydroquinone | | |
| | diacetate + (E/Z)-(all-rac)- | diacetate | | |
| | phytyl benzoate | | | |
| N | 4-acetyloxy-2-(3′-methyl-2′- | (E/Z)-4-acetyloxy-2- | 60% | 2.8 |
| | butenyl)-3,5,6-trimethylphenyl | phytyl-3,5,6- | | |
| | tributylsilyl ether + 2,6,10,14- | trimethylphenyl tributyl- | | |
| | tetramethylpentadecene | silyl ether | | |
| O | 4-acetyloxy-2-(3′-methyl-2′- | (E/Z)-4-acetyloxy-2- | 70% | 2.7 |
| | butenyl)-3,5,6-trimethylphenyl | phytyl-3,5,6- | | |
| | tert-butyldimethylsilyl ether + | trimethylphenyl tert- | | |
| | 2,6,10,14-tetramethylpentadecene | butyldimethylsilyl ether | | |
| P | 4-acetyloxy-2-(3′-methyl-2′- | (E/Z)-4-acetyloxy-2- | 56% | 2.6 |
| | butenyl)-3,5,6-trimethylphenyl | phytyl-3,5,6- | | |
| | tert-butyldimethylsilyl ether + | trimethylphenyl tert- | | |
| | 2,6,10,14-tetramethylpentadecene | butyldimethylsilyl ether | | |
| | in vacuo | | | |
| Q | 4-acetyloxy-2-(3′-methyl-2′- | (E/Z)-4-acetyloxy-2- | 52% | 2.1 |
| | butenyl)-3,5,6-trimethylphenyl | phytyl-3,5,6- | | |
| | tert-butyldimethylsilyl ether + | trimethylphenyl tert- | | |
| | (E/Z)-(all-rac)-phytyl acetate | butyldimethylsilyl ether | | |
| R | 4-acetyloxy-2-(3′-methyl-2′- | (E/Z)-4-acetyloxy-2- | 54% | 1.9 |
| | butenyl)-3,5,6-trimethylphenyl | phytyl-3,5,6- | | |
| | tert-butyldimethylsilyl ether + | trimethylphenyl tert- | | |
| | (E)-(R,R)-phytyl acetate | butyldimethylsilyl ether | | |
| S | (E/Z)-4-acetyloxy-2-(3′- | (E/Z)-4-acetyloxy-2- | 42% | 2.1 |
| | methyl-2′-butenyl)-3,5,6- | phytyl-3,5,6- | | |
| | trimethylphenyl tert- | trimethylphenyl tert- | | |
| | butyldimethylsilyl ether + | butyldimethylsilyl ether | | |
| | (E/Z)-(all-rac)-phytyl formiate | | | |

### Example J

### Synthesis of (E/Z)-3-phytyl-2,5,6-trimethylhydroquinone diacetate starting from 3-(3′-methyl-2′-butenyl)-2,5,6-trimethylhydroquinone diacetate and 2,6,10,14-tetramethylpentadecene

A schlenk tube, placed under argon and equipped with a magnetic stirrer, was charged with 0.01 mmol (8.4 mg) of the catalyst, 0.2 mmol (36.8 mg) of tridecane and 2 mL of toluene. To this solution, a mixture of 0.2 mmol (60.8 mg) of 3-(3′-methyl-2′-butenyl)-2,5,6-trimethylhydroquinone diacetate and 0.4 mmol (107 mg) of 2,6,10,14-tetramethylpentadecene dissolved in 4 mL of toluene was added at 21 to 22° C. The resulting brown solution was stirred at 21 to 22° C. for 10 minutes and then at 80° C. for 18 hours. The progress of the reaction can be monitored by GC. After 18 hours the meanwhile orange solution was cooled to 21 to 22° C. and reduced in vacuo to afford a brown oil which was purified by column chromatography over silica gel using a mixture of diethylether and hexane (v/v=1:4) as eluent. 0.14 mmol (71 mg) of (E/Z)-3-phytyl-2,5,6-trimethylhydroquinone diacetate were isolated as a colorless oil with a E/Z ratio of 2.5 (determined by ¹H NMR) (yield: 69% based on 3-(3′-methyl-2′-butenyl)-2,5,6-trimethylhydroquinone diacetate).

### Example K

### Synthesis of (E/Z)-3-phytyl-2,5,6-trimethylhydroquinone diacetate starting from 3-(3′-methyl-2′-butenyl)-2,5,6-trimethylhydroquinone diacetate and 2,6,10,14-tetramethylpentadecene in vacuo

A mixture of 0.8 mmol of 3-(3′-methyl-2′-butenyl)-2,5,6-trimethylhydroquinone diacetate, 1.6 mmol of 2,6,10,14-tetramethylpentadecene and 0.04 mmol of the catalyst was vigorously stirred at 80° C. for 3 hours in vacuo (33 mbar). The yield was 60% based on 3-(3′-methyl-2′-butenyl)-2,5,6-trimethylhydroquinone diacetate. The E/Z ratio of the product, (E/Z)-3-phytyl-2,5,6-trimethylhydroquinone diacetate, was 2.3 (determined by ¹H NMR).

### Example L

### Synthesis of (E/Z)-3-phytyl-2,5,6-trimethylhydroquinone diacetate starting from 3-(3′-methyl-2′-butenyl)-2,5,6-trimethylhydroguinone diacetate and (E/Z)-(all-rac)-phytyl acetate

Example J was repeated under the same conditions except that 0.4 mmol of (E/Z)-(all-rac)-phytyl acetate were used instead of 0.4 mmol of 2,6,10,14-tetramethylpentadecene. The yield was 46% based on 3-(3′-methyl-2′-butenyl-2,5,6-trimethylhydroquinone diacetate. The E/Z ratio of the product, (E/Z)-3-phytyl-2,5,6-trimethylhydroquinone diacetate, was 2.1 (determined by ¹H NMR).

### Example M

### Synthesis of (E/Z)-3-phytyl-2,5,6-trimethylhydroquinone diacetate starting from 3-(3′-methyl-2′-butenyl)-2,5,6-trimethylhydroquinone diacetate and (E/Z)-(all-rac)-phytyl benzoate

Example J was repeated under the same conditions except that 0.4 mmol of (E/Z)-(all-rac)-phytyl benzoate were used instead of 0.4 mmol of 2,6,10,14-tetramethylpentadecene. The yield was 50% based on 3-(3′-methyl-2′-butenyl)-2,5,6-trimethylhydroquinone diacetate. The E/Z ratio of the product, (E/Z)-3-phytyl-2,5,6-trimethylhydroquinone diacetate, was 2.0 (determined by ¹H NMR).

### Example N

### Synthesis of (E/Z)-4-acetyloxy-2-phytyl-3,5,6-trimethylphenyl tributylsilyl ether starting from 4-acetyloxy-2(3′-methyl-2′-butenyl)-3,5,6-trimethylphenyl tributylsilyl ether and 2,6,10,14-tetramethylpentadecene

Example J was repeated under the same conditions except that 0.2 mmol or 4-acetyloxy-2-(3′-methyl-2′-butenyl)-3,5,6-trimethylphenyl tributylsilyl ether were used instead of 0.2 mmol of 3-(3′-methyl-2′-butenyl)-2,5,6-trimethylhydroquinone diacetate. The yield was 60% based on 4-acetyloxy-2-(3′-methyl-2′-butenyl)-3,5,6-trimethylphenyl tributylsilyl ether. The E/Z ratio of the product, (E/Z)-4-acetyloxy-2-phytyl-3,5,6-trimethylphenyl tributylsilyl ether, was 2.8 (determined by GC).

### Example O

### Synthesis of (E/Z)-4-acetyloxy-2-phytyl-3,5,6-trimethylphenyl tert-butyldimethylsilyl ether starting from 4-acetyloxy-2-(3′-methyl-2′-butenyl)-3,5,6-trimethylphenyl tert-butyldimethylsilyl ether and 2,6,10,14-tetramethylpentadecene

Example J was repeated under the same conditions except that 0.2 mmol of 4-acetyloxy-2-(3′-methyl-2′-butenyl)-3,5,6-trimethylphenyl tert-butyldimethylsilyl ether were used instead of 0.2 mmol of 3-(3′-methyl-2′-butenyl)-2,5,6-trimethylhydroquinone diacetate. The yield was 70% based on 4-acetyloxy-2-(3′-methyl-2′-butenyl)-3,5,6-trimethylphenyl tert-butyldimethylsilyl ether. The E/Z ratio of the product, (E/Z)-4-acetyloxy-2-phytyl-3,5,6-trimethylphenyl tert-butyldimethylsilyl ether, was 2.7 (determined by GC).

### Example P

### Synthesis of (E/Z)-4-acetyloxy-2-phytyl-3,5,6-trimethylphenyl tert-butyldimethylsilyl ether starting from 4-acetyloxy-2-(3′-methyl-2′-butenyl)-3,5,6-trimethylphenyl tert-butyldimethylsilyl ether and 2,6,10,14-tetramethylpentadecene in vacuo

A mixture of 0.4 mmol of 4-acetyloxy-2-(3′-methyl-2′-butenyl)-3,5,6-trimethylphenyl tert-butyldimethylsilyl ether, 0.8 mmol of 2,6,10,14-tetramethylpentadecene and 0.02 mmol of the catalyst was vigorously stirred at 80° C. for 3 hours in vacuo (33 mbar). The yield was 56% based on 4-acetyloxy-2-(3′-methyl-2′-butenyl)-3,5,6-trimethylphenyl tert-butyldimethylsilyl ether. The E/Z ratio of the product, (E/Z)-4-acetyloxy-2-phytyl-3,5,6-trimethylphenyl tert-butyldimethylsilyl ether, was 2.6 (determined by GC).

### Example O

### Synthesis of (E/Z)-4-acetyloxy-2-phytyl-3,5,6-trimethylphenyl tert-butyldimethylsilyl ether starting from 4-acetyloxy-2-(3′-methyl-2′-butenyl)-3,5,6-trimethylphenyl tert-butyldimethylsilyl ether and (E/Z)-(all-rac)-phytyl acetate

Example O was repeated under the same conditions except that 0.4 mmol of (E/Z)-(all-rac)-phytyl acetate were used instead of 0.4 mmol of 2,6,10,14-tetramethylpentadecene. The yield was 52% based on 4-acetyloxy-2-(3′-methyl-2′-butenyl)-3,5,6-trimethylphenyl tert-butyldimethylsilyl ether. The E/Z ratio of the product, (E/Z)-4-acetyloxy-2-phytyl-3,5,6-trimethylphenyl tert-butyldimethylsilyl ether, was 2.1 (determined by GC).

### Example R

### Synthesis of (E/Z)-4-acetyloxy-2-phytyl-3,5,6-trimethylphenyl tert-butyldimethylsilyl ether starting from 4-acetyloxy-2-(3′-methyl-2′-butenyl)-3,5,6-trimethylphenyl tert-butyldimethylsilyl ether and (E)-(R,R)-phytyl acetate

Example O was repeated under the same conditions except that 0.4 mmol of (E)-(R,R)-phytyl acetate were used instead of 0.4 mmol of 2,6,10,14-tetramethylpentadecene. The yield was 54% based on 4-acetyloxy-2-(3′-methyl-2′-butenyl)-3,5,6-trimethylphenyl tert-butyldimethylsilyl ether. The E/Z ratio of the product, (E/Z)-4-acetyloxy-2-phytyl-3,5,6-trimethylphenyl tert-butyldimethylsilyl ether, was 1.9 (determined by GC).

### Example S

### Synthesis of (E/Z)-4-acetyloxy-2-phytyl-3,5,6-trimethylphenyl tert-butyldimethylsilyl ether starting from (E/Z)-4-acetyloxy-2-(3′-methyl-2′-butenyl)-3,5,6-trimethylphenyl tert-butyldimethylsilyl ether and (E/Z)-(all-rac)-phytyl formiate

Example O was repeated under the same conditions except that 0.4 mmol of (E)-(all-rac)-phytyl formiate were used instead of 0.4 mmol of 2,6,10,14-tetramethylpentadecene. The yield was 42% based on 4-acetyloxy-2-(3′-methyl-2′-butenyl)-3,5,6-trimethylphenyl tert-butyldimethylsilyl ether. The E/Z ratio of the product, (E/Z)-4-acetyloxy-2-phytyl-3,5,6-trimethylphenyl tert-butyldimethylsilyl ether, was 2.1 (determined by GC).

### Example T

### Synthesis of (E/Z)-3-phytyl-2,5,6-trimethylhydroquinone 1-acetate starting from (E/Z)-4-acetyloxy-2-phytyl-3,5,6-trimethylphenyl tert-butyldimethylsilyl ether

(See S. V. Ankala, G. Fenteany, Tetrahedron Lett. 2002, 43, 4729-4732.)

A mixture of 0.100 mmol (59.0 mg) of (E/Z)-4-acetyloxy-2-phytyl-3,5,6-trimethylphenyl tert-butyldimethylsilyl ether (E/Z=2.1) and 0.359 mmol (15.0 mg) of LiOH.H₂O in 0.2 mL of dimethylformamide was vigorously stirred at 22-23° C. during 16 hours. Then, the solvent was removed in vacuo and the crude oil was purified by column chromatography over silica gel using a mixture of diethylether and hexane (v/v=1:4) as eluent 0.066 mmol (31.2 mg) of (E/Z)-4-acetyloxy-2-phytyl-3,5,6-trimethylphenyl tert-butyldimethylsilyl ether were isolated as a colorless oil with an E/Z ratio of 2.1 (determined by GC) (yield: 66%-based on (E/Z)-4-acetyloxy-2-phytyl-3,5,6-trimethylphenyl tert-butyldimethylsilyl ether).

### Example U

### Synthesis of (E/Z)-3-phytyl-2,5,6-trimethylhydroquinone 1-acetate starting from (E/Z)-4-acetyloxy-2-phytyl-3,5,6-trimethylphenyl tributylsilyl ether

(See S. V. Ankala, G. Fenteany, Tetrahedron Lett. 2002, 43, 4729-4732.)

A mixture of 0.074 mmol (50.0 mg) of (E/Z)-4-acetyloxy-2-phytyl-3,5,6-trimethylphenyl tributylsilyl ether (E/Z=2.7) and 0.223 mmol (9.4 mg) of LiOH.H₂O in 0.2 mL of dimethylformamide was vigorously stirred at 22-23° C. during 16 hours. Then, the solvent was removed in vacuo and the crude oil was purified by column chromatography over silica gel using a mixture of diethylether and hexane (v/v=1:4) as eluent. 0-055 mmol (26.1 mg) of (E/Z)-4-acetyloxy-2-phytyl-3,5,6-trimethylphenyl tributylsilyl ether-were isolated as a colorless oil with an E/Z ratio of 2.5 (determined by GC) (yield: 74%-based on (E/Z)-4-acetyloxy-2-phytyl-3,5,6-trimethylphenyl tributylsilyl ether).

1. A process for the manufacture of compounds represented by the following formula III 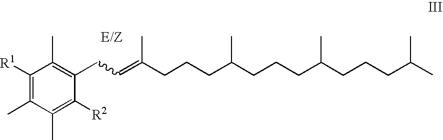
wherein R¹ is C₂₋₅-alkanoyloxy, and R² is C₂₋₅-alkanoyloxy or OSiR⁶R⁷R⁸, and wherein R⁶, R⁷ and R⁸ are independently from each other C₁₋₆-alkyl or phenyl,
by the reaction of
a) a compound represented by the following formula I 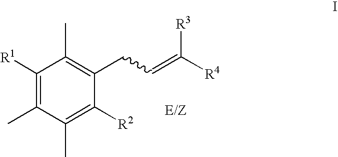
wherein R¹ and R² are as defined above, and
wherein R³ and R⁴ are independently from each other H or C₁₋₅-alkyl, with the proviso that at least one of R³ and R⁴ is not H, with
b) a compound represented by the following formula II 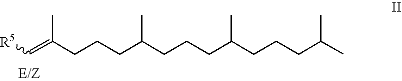
wherein R⁵ is H or CH₂—R⁹,
wherein R⁹ is formyloxy, C₂₋₅-alkanoyloxy, benzoyloxy, C₁₋₅-alkoxy or OSiR⁶R⁷R⁸ as defined above,
in the presence of a cross-metathesis catalyst.

2. The process as claimed in claim 1, wherein the cross-metathesis catalyst is a ruthenium compound used in homogeneous catalysis.

3. The process as claimed in claim 2, wherein the ruthenium compound is a ruthenium metal carbene complex possessing (a) ruthenium metal center(s), having an electron count of 16 and being penta-coordinated or a ruthenium metal carbene complex possessing (a) ruthenium metal center(s), having an electron count of 18 and being hexa-coordinated, preferably a ruthenium metal carbene complex possessing a ruthenium metal center, having an electron count of 16 and being penta-coordinated.

4. The process as claimed in claim 2, wherein the ruthenium compound is one of the complexes represented by the following formulae VIIa, VIIb and VIIc: 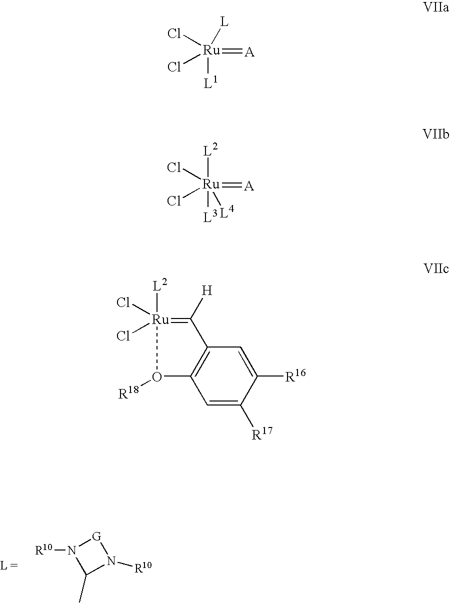
wherein R¹⁰ is an optionally single or multiple C₁₋₅-alkylated and/or C₁₋₅-alkoxylated phenyl,
G is ethane-1,2-diyl, ethylene-1,2-diyl, cyclohexane-1,2-diyl or 1,2-diphenylethane-1,2-diyl,
L¹ is PR¹¹R¹²R¹³,
wherein R¹¹, R¹² and R¹³ are independently from each other C₁₋₈-alkyl, phenyl or tolyl,
A is CH₂, C(H)aryl, C(H)R⁴, C═C(R⁴)₂, C═C(H)Si(R⁵)₃, C(H)—C(H)═C(R¹⁴)₂, C═C(H)(phenyl), C(H)—C(H)═C(phenyl)₂ or C═C═C(phenyl)₂,
wherein “aryl” is an optionally single or multiple C₁₋₅-alkylated and/or halogenated phenyl, R¹⁴ is C₁₋₄-alkyl, R¹⁵ is C₁₋₆-alkyl or phenyl,
L² is L or L:
L³ and L⁴ are independently from each other pyridyl or 3-halopyridyl, wherein halo is Br or C¹,
R¹⁶ and R¹⁷ are both H or form together a fused benzene ring, and R¹⁸ is C₁₋₅-alkoxy.

5. The process as claimed in claim 2, wherein the ruthenium compound is represented by the following formula VIII

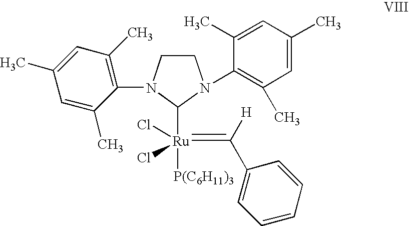

6. The process as claimed in claim 1, wherein the reaction is carried out in an aprotic organic solvent.

7. The process as claimed in claim 6, wherein the aprotic organic solvent is a dialkyl ether R¹⁹—O—R²⁰, tetrahydrofuran, tetrahydropyran, 1,4-dioxane, methylene chloride, chloroform, cumene, an optionally once, twice or thrice methylated arylene, or a mixture thereof,
wherein R¹⁹ and R²⁰ are independently from each other linear C₁₋₄-alkyl or branched C₃₋₈-alkyl.

8. The process as claimed in claim 7, wherein the aprotic organic solvent is tetrahydrofuran, methylene chloride, chloroform, toluene or a mixture thereof, preferably toluene.

9. The process as claimed in claim 6, wherein from about 3 ml to about 15 ml, preferably from about 4 ml to about 10 ml, more preferably from about 4.5 ml to about 8 ml of the aprotic organic solvent are used per mmol of compound a) or b), whichever is used in the lesser amount.

10. The process as claimed in claim 1, wherein the reaction is carried out essentially in the absence of an additional solvent.

11. The process as claimed in claim 10, wherein the reaction is carried out in vacuo, preferably at a pressure below 100 mbar.

12. The process as claimed in claim 1, wherein the relative amount of the cross-metathesis catalyst to the amount of compound a) or b), whichever is used in the lesser amount, is from about 0.0001 mol % to about 20 mol %, preferably from about 1.0 mol % to about 10 mol %, more preferably from about 2 to about 5 mol %.

13. The process according to claim 1, wherein the molar ratio of compound a) to compound b) present in the reaction mixture is from about 1:10 to about 10:1, preferably from about 1:5 to about 5:1, more preferably from about 1:3 to about 1:2.5.

14. The process as claimed in claim 1 wherein the reaction is carried out at temperatures from about 10° C. to about 120° C., preferably from about 30° C. to about 100° C., especially from about 40° C. to about 85° C.

15. A process for the manufacture of α-tocopherol and α-tocopheryl alkanoates represented by the following formula V 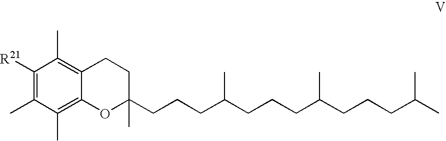
comprising the following steps:
i) reacting of a compound represented by the following formula I 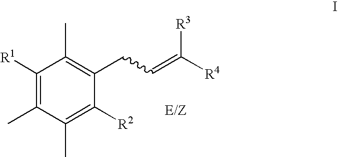
with a compound represented by the following formula II 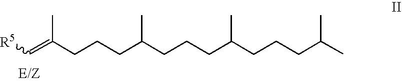
to a compound represented by the following formula III 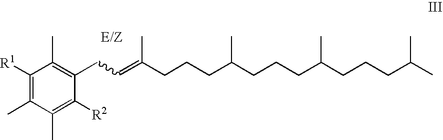
in the presence of a cross-metathesis catalyst,
ii) converting the compound represented by the formula III and obtained in step i) to (E/2)-3-phytyl-2,5,6-trimethylhydroquinone or a (E/Z)-3-phylyl-2,5,6-trimethylhydroquinone 1-alkanoate represented by the following formula IV, and 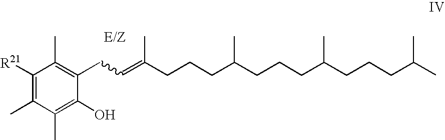
iii) subjecting the (E/Z)-3-phytyl-2,5,6-trimethylhydroquinone or (E/Z)-3-phytyl-2,5,6-trimethylhydroquinone 1-alkanoate represented by the formula IV and obtained in step ii) to a cyclization to α-tocopherol or an α-tocopheryl alkanoate represented by the formula V,
wherein R¹, R², R³, R⁴ and R⁵ are as defined in claim 1, and R²¹ is R¹ or OH.

16. Compounds of the formula III 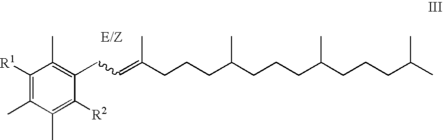
wherein R¹ is C₂₋₅-alkanoyloxy, and R² is C₂₋₅-alkanoyloxy or OSiR⁶R⁷R⁸, and wherein R⁶, R⁷ and R⁸ are independently from each other C₁₋₆-alkyl or phenyl.

17. Compounds of the formula IX 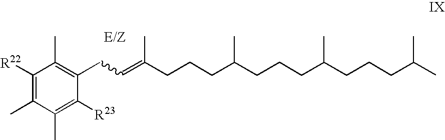
wherein R²² is C₃₋₅-alkanoyloxy, and R²³ is C₃₋₅-alkanoyloxy or OSiR⁶R⁷R⁸, and wherein R⁶, R⁷ and R⁸ are independently from each other C₁₋₆-alkyl or phenyl.

18. Compounds of the formula X 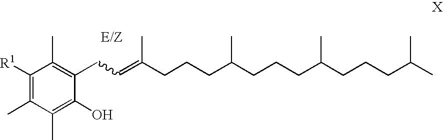
wherein R¹ is C₂₋₅-alkanoyloxy.

19. Compounds of the formula XI 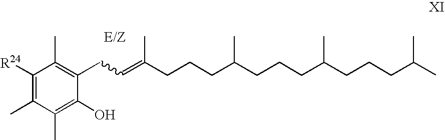
wherein R²⁴ is C₃₋₅-alkanoyloxy.
